(19) United States
(12) Patent Application Publication
(10) Pub. No.: US 2024/0335579 A1
(43) Pub. Date: Oct. 10, 2024

(54) Shopping Cart Sanitizing Assembly

(71) Applicant: Holly Baldwin, Tyler, TX (US)

(72) Inventor: Holly Baldwin, Tyler, TX (US)

(21) Appl. No.: 18/747,593

(22) Filed: Jun. 19, 2024

Related U.S. Application Data
(63) Continuation-in-part of application No. 17/476,777, filed on Sep. 16, 2021, now Pat. No. 12,043,226.

(51) Int. Cl. A61L 2/10; A61L 2/24; A61L 2/26

(52) U.S. Cl. CPC A61L 2/10; A61L 2/24; A61L 2/26; A61L 2202/11; A61L 2202/14

(57) ABSTRACT

A shopping cart sanitizing assembly includes a tunnel that is positionable in a location which employs shopping carts wherein the tunnel is configured to have the shopping carts moved through the tunnel. A plurality of lower ultraviolet lights and a plurality of upper ultraviolet lights is each integrated into the tunnel to project ultraviolet light for sanitizing shopping carts. A first drive unit and a second drive unit is each movably integrated into the tunnel. A plurality of grappling units each extends between the first drive unit and the second drive unit. Each of the grapping units engages a handle of a respective shopping cart to transport the respective shopping cart through the tunnel.

### (b) CROSS-REFERENCE TO RELATED APPLICATIONS

This application is a continuation in part of U.S. application Ser. No. 17/476,777 filed on Sep. 16, 2021 and claims the benefit thereof under Title 35, United States Code, Section 120.

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to sanitizing devices and more particularly pertains to a new sanitizing device for automatically sanitizing shopping carts. The device includes a tunnel with a pair of drive belts extending through the tunnel. A plurality of grappling units is coupled between the drive belts and each of the grappling units engages a handle of a shopping cart. A plurality of ultraviolet lights is integrated into the tunnel for sanitizing the shopping carts as the shopping carts are transported through the tunnel.

### (2) DESCRIPTION OF RELATED ART INCLUDING INFORMATION DISCLOSED UNDER 37 CFR 1.97 AND 1.98

The prior art relates to sanitizing devices including a variety of automated shopping cart sanitizing devices that include a tunnel and a plurality of sprayers integrated into the tunnel for automatically washing shopping carts. The prior art discloses a variety of automated washing devices that includes a transporting unit for transporting an object through an elongated washing area.

### (h) BRIEF SUMMARY OF THE INVENTION

An embodiment of the disclosure meets the needs presented above by generally comprising a tunnel that is positionable in a location which employs shopping carts wherein the tunnel is configured to have the shopping carts moved through the tunnel. A plurality of lower ultraviolet lights and a plurality of upper ultraviolet lights is each integrated into the tunnel to project ultraviolet light for sanitizing shopping carts. A first drive unit and a second drive unit is each movably integrated into the tunnel. A plurality of grappling units each extends between the first drive unit and the second drive unit. Each of the grapping units engages a handle of a respective shopping cart to transport the respective shopping cart through the tunnel.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a right side phantom view of a shopping cart sanitizing assembly according to an embodiment of the disclosure.

FIG. 2 is a back phantom view of an embodiment of the disclosure.

FIG. 3 is a cross sectional view taken along line 3-3 of FIG. 2 of an embodiment of the disclosure.

### (j) DETAILED DESCRIPTION OF THE INVENTION

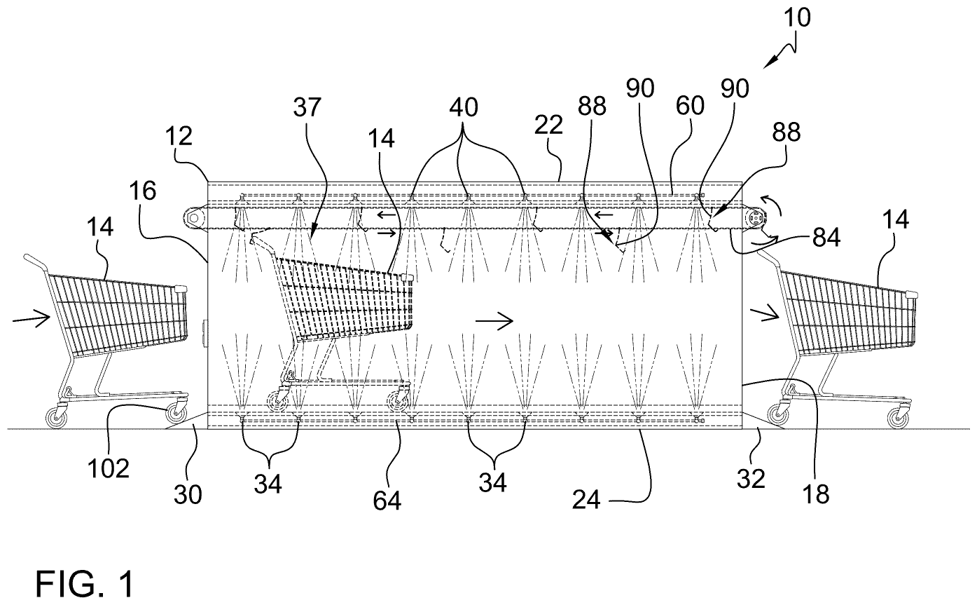

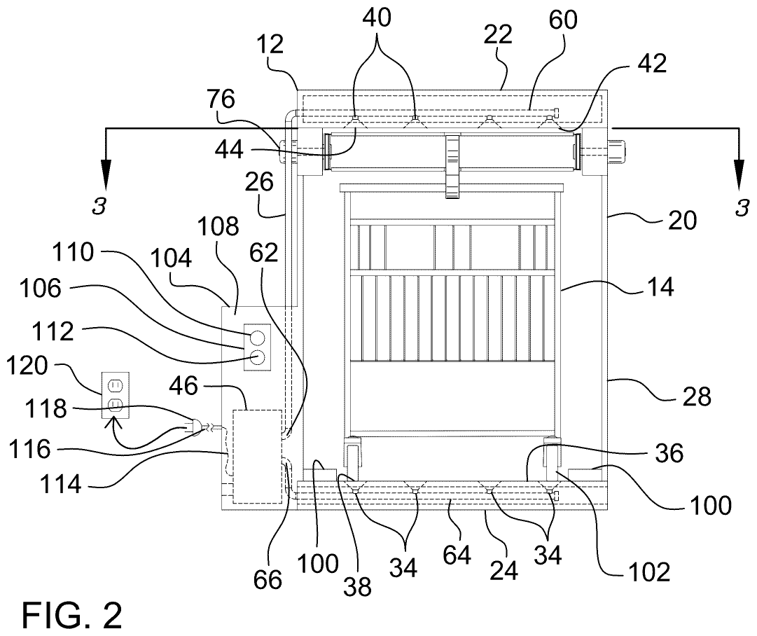

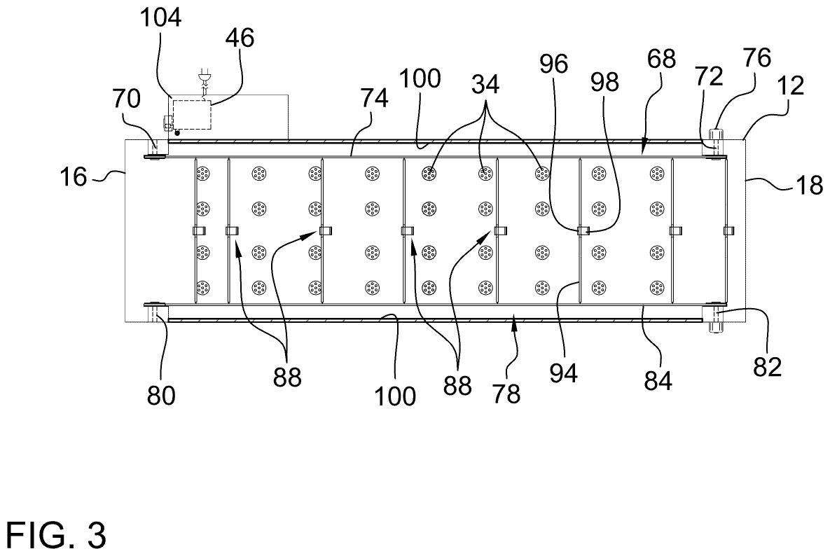

With reference now to the drawings, and in particular to FIGS. 1 through 3 thereof, a new sanitizing device embodying the principles and concepts of an embodiment of the disclosure and generally designated by the reference numeral 10 will be described.

As best illustrated in FIGS. 1 through 3, the shopping cart sanitizing assembly generally comprises a tunnel 12 that is positionable in a location which employs shopping carts 14 for having the shopping carts 14 being moved through the tunnel 12. The tunnel 12 has a first end 16, a second end 18 and an outer wall 20 extending between the first end 16 and the second end 18. Each of the first end 16 and the second end 18 is open, and the outer wall 20 has a top side 22, a bottom side 24, a first lateral side 26 and a second lateral side 28. A first ramp 30 is positioned against the first end 16 of the tunnel 12 and the first ramp 30 slopes upwardly into the bottom side 24 of the outer wall 20 of the tunnel 12 to facilitate the shopping carts 14 to roll into the tunnel 12. A second ramp 32 is positioned against the second end 18 of the tunnel 12 and the second ramp 32 slopes upwardly into the bottom side 24 of the outer wall 20 of the tunnel 12 to facilitate the shopping carts 14 to roll into the tunnel 12.

A plurality of lower ultraviolet lights 34 is each integrated into a floor 36 of the tunnel 12 to project ultraviolet light 37 upwardly from the floor 36 for sanitizing shopping carts 14 when the shopping carts 14 are inside of the tunnel 12. Each of the lower ultraviolet lights 34 is embedded into the bottom side 24 of the outer wall 20, and the lower ultraviolet lights 34 are spaced apart from each other and are distributed between the first end 16 and the second end 18. The lower ultraviolet lights 34 are arranged into a plurality of rows that are distributed between the first lateral side 26 and the second lateral side 28 of the outer wall 20 of the tunnel 12. Each of the lower ultraviolet lights 34 has an open end 38 that is exposed with respect to the bottom side 24 of the outer wall 20. Additionally, the open end 38 is open to project the ultraviolet light 37 upwardly from the bottom side 24 of the outer wall 20.

A plurality of upper ultraviolet lights 40 is provided and each of the upper ultraviolet lights 40 is integrated into a ceiling 42 of the tunnel 12 to project ultraviolet light 37 downwardly from the ceiling 42 for sanitizing shopping carts 14 when the shopping carts 14 are inside of the tunnel 12. Each of the upper ultraviolet lights 40 is embedded into the top side 22 of the outer wall 20. Additionally, the upper ultraviolet lights 40 are spaced apart from each other and are distributed between the first end 16 and the second end 18. The upper ultraviolet lights 40 are arranged into a plurality of rows that are distributed between the first lateral side 26 and the second lateral side 28 of the outer wall 20 of the tunnel 12. Each of the upper ultraviolet lights 40 has an open end 44 that is exposed with respect to the top side 22 of the outer wall 20. The open end 44 of each of the upper ultraviolet lights 40 is open to project ultraviolet light 37 upwardly from the bottom side 24 of the outer wall 20.

A power source 46 is provided and the power source 46 is coupled to the tunnel 12. The power source 46 is in electric communication with each of the lower ultraviolet lights 34 and each of the upper ultraviolet lights 40.

A plurality of upper conduits 60 is provided and each of the upper conduits 60 is coupled to a respective one of the upper ultraviolet lights 40 for holding wiring to electrically couple the power source 46 to each of the upper ultraviolet lights 40. The plurality of upper conduits 60 has a common inlet 62 coupled to the power source 46. In this way the upper conduits 60 can direct the wiring to each of the upper ultraviolet lights 40. A plurality of lower conduits 64 is each coupled to a respective one of the lower ultraviolet lights 34. The plurality of lower conduits 64 has a common inlet 66 coupled to the power source 46 to direct wiring to each of the lower ultraviolet lights 34.

A first drive unit 68 is movably integrated into the tunnel 12 and the first drive unit 68 extends along a full length of the tunnel 12. The first drive unit 68 moves in a first direction when the first drive unit 68 is turned on. The first drive unit 68 comprises a first roller 70 that is rotatably positioned on the first end 16 of the tunnel 12. The first roller 70 is positioned adjacent to an intersection between the top side 22 and the first lateral side 26 of the outer wall 20 of the tunnel 12. The first drive unit 68 includes a second roller 72 that is rotatably positioned on the second end 18 of the tunnel 12. The second roller 72 is positioned adjacent to an intersection between the top side 22 and the second lateral side 28 of the outer wall 20 of the tunnel 12.

The first drive unit 68 includes a first belt 74 that extends around each of the first roller 70 and the second roller 72. Additionally, the first drive unit 68 includes a first motor 76 that is coupled to the first roller 70. The first motor 76 rotates the first roller 70 in a first direction when the first motor 76 is turned on. The first motor 76 may comprise an electric motor or the like.

A second drive unit 78 is movably integrated into the tunnel 12 and the second drive unit 78 extends along a full length of the tunnel 12. The second drive unit 78 moves in a first direction when the second drive unit 78 is turned on. The second drive unit 78 comprises a primary roller 80 that is rotatably positioned on the first end 16 of the tunnel 12. The primary roller 80 is positioned adjacent to an intersection between the top side 22 and the second lateral side 28 of the outer wall 20 of the tunnel 12. The second drive unit 78 includes a secondary roller 82 that is rotatably positioned on the second end 18 of the tunnel 12. The secondary roller 82 is positioned adjacent to an intersection between the top side 22 and the second lateral side 28 of the outer wall 20 of the tunnel 12.

The second drive unit 78 includes a second belt 84 that extends around each of the primary roller 80 and the secondary roller 82. The second drive unit 78 includes a second motor 86 that is coupled to the secondary roller 82. Additionally, the second motor 86 rotates the secondary roller 82 in a first direction when the second motor 86 is turned on. The second motor 86 may comprise an electric motor or the like.

A plurality of grappling units 88 is provided and each of the grappling units 88 extends between the first drive unit 68 and the second drive unit 78. Each of the grappling units 88 has an engagement 90 extending downwardly to engage a handle 92 of a respective shopping cart 14. Additionally, each of the grappling units 88 is transported along the tunnel 12 when the first drive unit 68 and the second drive unit 78 are turned on. In this way each of the grappling units 88 can transport the respective shopping cart 14 through the tunnel 12. The grappling units 88 are spaced apart from each other and are distributed along a full length of the first drive unit 68 and the second drive unit 78.

Each of the grappling units 88 comprises a member 94 which extends between the first belt 74 and the second belt 84. The member 94 is comprised of a flexible material such as plastic, rubber or other similar material. Each of the grappling units 88 includes a hook 96 that is coupled to and extends downwardly from the member 94. The hook 96 has a distal end 98 with respect to the member 94 and the hook 96 is curved adjacent to the distal end 98 such that the distal end 98 is directed upwardly such that the hook 96 defines the engagement 90. Moreover, the hook 96 is oriented such that the distal end 98 is directed toward the second end 18 of the tunnel 12. A pair of guides 100 is positioned on the bottom side 24 of the outer wall 20 of the tunnel 12 and each of the guides 100 extends between the first end 16 and the second end 18 of the tunnel 12. Each of the guides 100 is positioned adjacent to a respective one of the first lateral side 26 and the second lateral side 28 of the outer wall 20 to guide wheels 102 on the shopping carts 14 when the shopping carts 14 are transported through the tunnel 12.

A power housing 104 is coupled to the outer wall 20 of the tunnel 12, and the power source 46 is positioned inside of the power housing 104. A control panel 106 is coupled to an outside wall 108 of the power housing 104 such that the control panel 106 is accessible to a user. The control panel 106 is in electrical communication with each of the first drive unit 68, the second drive unit 78 and the pump 46. Additionally, the control panel 106 controls operational parameters of each of the first drive unit 68, the second drive unit 78 and the power source 46 delivering electricity to each of the upper ultraviolet lights 40 and the lower ultraviolet lights 34.

The control panel 106 includes an on button 110 and an off button 112. The on button 110 turns on each of the first drive unit 68, the second drive unit 78 and the power source 46 when the on button is depressed. The off button 112 turns off each of the first drive unit 68, the second drive unit 78 and the power source 46 when the off button 112 is depressed. A power cord 114 is connected to the power source 46 and coupled to and extends away from the control panel 106. The power cord 114 is electrically coupled to the control panel 106. The power cord 114 has a distal end 116 with respect to the control panel 106 and a male plug 118 is electrically coupled to this distal end 116 that can be plugged into a female electrical outlet 120.

In use, the on button 110 on the control panel 106 is depressed and the shopping carts 14 are fed into the first end 16 of the tunnel 12 such that each of the grappling units 88 can engage the handle 92 of a respective shopping cart 14. In this way the shopping carts 14 are transported through the tunnel 12. Each of the upper ultraviolet lights 40 and the lower ultraviolet lights 34 sanitizes the shopping carts 14 as they are transported through the tunnel 12. In this way the shopping carts 14 are automatically sanitized.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A shopping cart sanitizing assembly for automatically sanitizing shopping carts, said assembly comprising:
a tunnel being positionable in a location which employs shopping carts wherein said tunnel is configured to have the shopping carts moved through said tunnel;
a plurality of lower ultraviolet lights, each of said lower ultraviolet lights being integrated into a floor of said tunnel wherein each of said lower ultraviolet lights is configured to project ultraviolet light upwardly from said floor for sanitizing shopping carts when the shopping carts are inside of said tunnel;
a plurality of upper ultraviolet lights, each of said upper ultraviolet lights being integrated into a ceiling of said tunnel wherein each of said upper ultraviolet lights is configured to project ultraviolet light downwardly from said ceiling for sanitizing shopping carts when the shopping carts are inside of said tunnel;
a first drive unit being movably integrated into said tunnel, said first drive unit extending along a full length of said tunnel, said first drive unit moving in a first direction when said first drive unit is turned on;
a second drive unit being movably integrated into said tunnel, said second drive unit extending along a full length of said tunnel, said second drive unit moving in a first direction when said second drive unit is turned on; and
a plurality of grappling units, each of said grappling units extending between said first drive unit and said second drive unit, each of said grapping units having an engagement extending downwardly wherein said engagement of each of said grapping units is configured to engage a handle of a respective shopping cart, each of said grapping units being transported along said tunnel when said first drive unit and said second drive unit are turned on wherein each of said grappling units is configured to transport the respective shopping cart through said tunnel.

2. The assembly according to claim 1, wherein:
said tunnel has a first end, a second end and an outer wall extending between said first end and said second end, each of said first end and said second end being open, said outer wall having a top side, a bottom side, a first lateral side and a second lateral side;
said assembly includes a first ramp being positioned against said first end of said tunnel, said first ramp sloping upwardly into said bottom side of said outer wall of said tunnel wherein said first ramp is configured to facilitate the shopping carts to roll into said tunnel;
said assembly includes a second ramp being positioned against said second end of said tunnel, said second ramp sloping upwardly into said bottom side of said outer wall of said tunnel wherein said second ramp is configured to facilitate the shopping carts to roll into said tunnel;
each of said lower ultraviolet lights is embedded into said bottom side of said outer wall, said lower ultraviolet lights being spaced apart from each other and being distributed between said first end and said second end, said lower ultraviolet lights being arranged into a plurality of rows being distributed between said first lateral side and said second lateral side of said outer wall of said tunnel, each of said lower ultraviolet lights having an open end being exposed with respect to said bottom side of said outer wall, said open end being open wherein said open end is configured to project the ultraviolet light upwardly from said bottom side of said outer wall; and
each of said upper ultraviolet lights is embedded into said top side of said outer wall, said upper ultraviolet lights being spaced apart from each other and being distributed between said first end and said second end, said upper ultraviolet lights being arranged into a plurality of rows being distributed between said first lateral side and said second lateral side of said outer wall of said tunnel, each of said upper spray ultraviolet lights having an open end being exposed with respect to said top side of said outer wall, said open end of each of said upper ultraviolet lights being open wherein said open end of each of said upper ultraviolet lights is configured to project ultraviolet light upwardly from said bottom side of said outer wall.

3. The assembly according to claim 2, further comprising:
a power source being coupled to said tunnel, said power source being in electrical communication with each of said lower ultraviolet lights and each of said upper ultraviolet lights;
said assembly includes a plurality of upper conduits, each of said upper conduits being coupled to a respective one of said upper ultraviolet lights, said plurality of upper conduits having a common inlet coupled to said power source wherein said upper conduits are configured to direct wiring to each of said upper ultraviolet lights; and
said assembly includes a plurality of lower conduits, each of said lower conduits being coupled to a respective one of said lower ultraviolet lights, said plurality of lower conduits having a common inlet coupled to said power source wherein said lower conduits are configured to direct wiring to each of said lower ultraviolet lights.

4. The assembly according to claim 2, wherein said first drive unit comprises:
a first roller being rotatably positioned on said first end of said tunnel, said first roller being positioned adjacent to an intersection between said top side and said first lateral side of said outer wall of said tunnel;
a second roller being rotatably positioned on said second end of said tunnel, said second roller being positioned adjacent to an intersection between said top side and said second lateral side of said outer wall of said tunnel;
a first belt extending around each of said first roller and said second roller; and
a first motor being coupled to said first roller such that said first motor rotates said first roller in a first direction when said first motor is turned on.

5. The assembly according to claim 4, wherein said second drive unit comprises:
a primary roller being rotatably positioned on said first end of said tunnel, said primary roller being positioned adjacent to an intersection between said top side and said second lateral side of said outer wall of said tunnel;
a secondary roller being rotatably positioned on said second end of said tunnel, said secondary roller being positioned adjacent to an intersection between said top side and said second lateral side of said outer wall of said tunnel;
a second belt extending around each of said primary roller and said secondary roller; and
a second motor being coupled to said secondary roller such that said second motor rotates said secondary roller in a first direction when said second motor is turned on.

6. The assembly according to claim 5, wherein each of said grappling units comprises:
a member extending between said first belt and said second belt, said member being comprised of a flexible material; and
a hook being coupled to and extending downwardly from said member, said hook having a distal end with respect to said member, said hook being curved adjacent to said distal end such that said distal end is directed upwardly wherein said hook defines said engagement, said hook being oriented such that said distal end is directed toward said second end of said tunnel.

7. The assembly according to claim 2, further comprising a pair of guides, each of said guides being positioned on said bottom side of said outer wall of said tunnel, each of said guides extending between said first end and said second end of said tunnel, each of said guides being positioned adjacent to a respective one of said first lateral side and said second lateral side of said outer wall wherein each of said guides is configured to guide wheels on the shopping carts when the shopping carts are transported through said tunnel.

8. The assembly according to claim 1, further comprising:
a power housing being coupled to an outer wall of said tunnel, said power source being positioned inside of said power housing;
a control panel being coupled to an outside wall of said power housing wherein said control panel is configured to be accessible to a user, said control panel being in electrical communication with each of said first drive unit, said second drive unit and said power source for delivering electricity to said upper ultraviolet lights and said lower ultraviolet lights, said control panel controlling operational parameters of each of said first drive unit, said second drive unit and said power source, said control panel including an on button and an off button, said on button turning on each of said first drive unit, said second drive unit and said power source when said on button is depressed, said off button turning off each of said first drive unit, said second drive unit and said power source when said off button is depressed; and
a power cord being coupled to and extending away from said control panel, said power cord being electrically coupled to said control panel, said power cord having a distal end with respect to said control panel, said distal end having a male plug being electrically coupled thereto wherein said male plug is configured to be plugged into a female electrical outlet.

9. A shopping cart sanitizing assembly for automatically sanitizing shopping carts, said assembly comprising:
a tunnel being positionable in a location which employs shopping carts wherein said tunnel is configured to have the shopping carts moved through said tunnel, said tunnel having a first end, a second end and an outer wall extending between said first end and said second end, each of said first end and said second end being open, said outer wall having a top side, a bottom side, a first lateral side and a second lateral side;
a first ramp being positioned against said first end of said tunnel, said first ramp sloping upwardly into said bottom side of said outer wall of said tunnel wherein said first ramp is configured to facilitate the shopping carts to roll into said tunnel;
a second ramp being positioned against said second end of said tunnel, said second ramp sloping upwardly into said bottom side of said outer wall of said tunnel wherein said second ramp is configured to facilitate the shopping carts to roll into said tunnel;
a plurality of lower ultraviolet lights, each of said lower ultraviolet lights being integrated into a floor of said tunnel wherein each of said lower ultraviolet lights is configured to project ultraviolet light upwardly from said floor for sanitizing shopping carts when the shopping carts are inside of said tunnel, each of said lower ultraviolet lights being embedded into said bottom side of said outer wall, said lower ultraviolet lights being spaced apart from each other and being distributed between said first end and said second end, said lower ultraviolet lights being arranged into a plurality of rows being distributed between said first lateral side and said second lateral side of said outer wall of said tunnel, each of said lower ultraviolet lights having an open end being exposed with respect to said bottom side of said outer wall, said open end being open wherein said open end is configured to project ultraviolet light upwardly from said bottom side of said outer wall;
a plurality of upper ultraviolet lights, each of said upper ultraviolet lights being integrated into a ceiling of said tunnel wherein each of said upper ultraviolet lights is configured to project ultraviolet light downwardly from said ceiling for sanitizing shopping carts when the shopping carts are inside of said tunnel, each of said upper ultraviolet lights being embedded into said top side of said outer wall, said upper ultraviolet lights being spaced apart from each other and being distributed between said first end and said second end, said upper ultraviolet lights being arranged into a plurality of rows being distributed between said first lateral side and said second lateral side of said outer wall of said tunnel, each of said upper ultraviolet lights having an open end being exposed with respect to said top side of said outer wall, said open end of each of said upper ultraviolet lights being open wherein said open end of each of said upper ultraviolet lights is configured to project ultraviolet light upwardly from said bottom side of said outer wall;
a power source being coupled to said tunnel, said power source being in electrical communication with each of said lower ultraviolet lights and each of said upper ultraviolet lights, said power source having an upper output and a lower output;
a plurality of upper conduits, each of said upper conduits being fluidly coupled to a respective one of said upper ultraviolet lights, said plurality of upper conduits having a common inlet coupled to said power source wherein said upper conduits are configured to direct wiring to each of said upper ultraviolet lights;
a plurality of lower conduits, each of said lower conduits being coupled to a respective one of said lower ultraviolet lights, said plurality of lower conduits having a common inlet coupled to said power source wherein said lower conduits are configured to direct wiring to each of said lower ultraviolet lights;
a first drive unit being movably integrated into said tunnel, said first drive unit extending along a full length of said tunnel, said first drive unit moving in a first direction when said first drive unit is turned on, said first drive unit comprising:
a first roller being rotatably positioned on said first end of said tunnel, said first roller being positioned adjacent to an intersection between said top side and said first lateral side of said outer wall of said tunnel;
a second roller being rotatably positioned on said second end of said tunnel, said second roller being positioned adjacent to an intersection between said top side and said second lateral side of said outer wall of said tunnel;
a first belt extending around each of said first roller and said second roller; and
a first motor being coupled to said first roller such that said first motor rotates said first roller in a first direction when said first motor is turned on;
a second drive unit being movably integrated into said tunnel, said second drive unit extending along a full length of said tunnel, said second drive unit moving in a first direction when said second drive unit is turned on, said second drive unit comprising:
a primary roller being rotatably positioned on said first end of said tunnel, said primary roller being positioned adjacent to an intersection between said top side and said second lateral side of said outer wall of said tunnel;
a secondary roller being rotatably positioned on said second end of said tunnel, said secondary roller being positioned adjacent to an intersection between said top side and said second lateral side of said outer wall of said tunnel;
a second belt extending around each of said primary roller and said secondary roller; and
a second motor being coupled to said secondary roller such that said second motor rotates said secondary roller in a first direction when said second motor is turned on;
a plurality of grappling units, each of said grappling units extending between said first drive unit and said second drive unit, each of said grapping units having an engagement extending downwardly wherein said engagement of each of said grapping units is configured to engage a handle of a respective shopping cart, each of said grapping units being transported along said tunnel when said first drive unit and said second drive unit are turned on wherein each of said grappling units is configured to transport the respective shopping cart through said tunnel, each of said grappling units comprising:
a member extending between said first belt and said second belt, said member being comprised of a flexible material; and
a hook being coupled to and extending downwardly from said member, said hook having a distal end with respect to said member, said hook being curved adjacent to said distal end such that said distal end is directed upwardly wherein said hook defines said engagement, said hook being oriented such that said distal end is directed toward said second end of said tunnel;
a pair of guides, each of said guides being positioned on said bottom side of said outer wall of said tunnel, each of said guides extending between said first end and said second end of said tunnel, each of said guides being positioned adjacent to a respective one of said first lateral side and said second lateral side of said outer wall wherein each of said guides is configured to guide wheels on the shopping carts when the shopping carts are transported through said tunnel;
a power housing being coupled to said outer wall of said tunnel, said power source being positioned inside of said power housing;
a control panel being coupled to an outside wall of said power housing wherein said control panel is configured to be accessible to a user, said control panel being in electrical communication with each of said first drive unit, said second drive unit and said power source for delivering electricity to said upper ultraviolet lights and said lower ultraviolet lights, said control panel controlling operational parameters of each of said first drive unit, said second drive unit and said power source, said control panel including an on button and an off button, said on button turning on each of said first drive unit, said second drive unit and said power source when said on button is depressed, said off button turning off each of said first drive unit, said second drive unit and said power source when said off button is depressed; and
a power cord being coupled to and extending away from said control panel, said power cord being electrically coupled to said control panel, said power cord having a distal end with respect to said control panel, said distal end having a male plug being electrically coupled thereto wherein said male plug is configured to be plugged into a female electrical outlet.
